(19) United States
(12) Patent Application Publication
(10) Pub. No.: US 2020/0107767 A1
(43) Pub. Date: Apr. 9, 2020

(54) Non-Invasive Portable Device and Method to Assess Mental Conditions

(71) Applicant: Synapstory Production Group Inc., Washington, DC (US)

(72) Inventor: Wendy ANSON, Washington, DC (US)

(21) Appl. No.: 16/595,664

(22) Filed: Oct. 8, 2019

Related U.S. Application Data
(60) Provisional application No. 62/743,300, filed on Oct. 9, 2018.

(51) Int. Cl. A61B 5/16; G16H 50/20

(52) U.S. Cl. CPC A61B 5/165; G16H 50/20; A61B 5/168; A61B 5/163

(57) ABSTRACT

A system of hardware and software that captures and processes in real time biometric data of patient responses and reactions to tablet-based valenced pictorial stimuli to facilitate diagnosis of mental health conditions of the patient including Autism Spectrum Disorder (ASD), Attention-Deficit Hyperactivity Disorder (ADHD), Traumatic Brain Injury (TBI), and Post-Traumatic Stress Disorder (PTSD) is provided. Via transparent, non-invasive biometrics induced by pictorial stimuli, the system removes potentially threatening testing instrument characteristics that can invalidate authentic assessment. The system will mitigate or remove other threats to assessment validity including subjective judgments as well as bias on the part of the examiners who are completing subjective surveys. The biometric assessment is incorporated within a variety of engaging game formats that appeal to males and females of nearly any age that speak a variety of languages and encompass a wide spectrum of demographics and ethnicities.

### BACKGROUND AND SUMMARY OF THE INVENTION

The invention relates to a non-invasive device and method to identify dysfunctional mental conditions (e.g., Post-Traumatic Stress Disorder (PTSD), Autism Spectrum Disorder (ASD), Attention-Deficit Hyperactivity Disorder (ADHD), Traumatic Brain Injury (TBI)) in children and adults via facial expression, heart rate variability (HRV) and eye movement-driven biometrics, among other metrics resulting from research-based stimuli.

PTSD, ASD, ADHD, and TBI are dysfunctional conditions or states of the brain whose characteristics include significantly different amounts of attentional focus, facial expression, anxiety, and/or apparent memory deficits than is the case in non-pathological brain states. ASD is a neurodevelopmental disorder characterized by repetitive and characteristic patterns of behavior and difficulties with social communication and interaction including abnormally intense or focused interest, preoccupation with certain objects or subjects, lack of smiling, repetitive or unusual use of language. ADHD is a brain disorder marked by an ongoing pattern of inattention and/or hyperactivity-impulsivity that interferes with functioning or development. TBI is a form of acquired brain injury with symptoms that can include confusion, blurred vision, fatigue or lethargy, behavioral or mood changes, dilation of one or both pupils of the eyes, poor coordination, and increased confusion, restlessness, or agitation and trouble with memory, concentration, attention or thinking.

According to the National Institutes of Health (NIH), PTSD is a mental health problem that some people develop after experiencing or witnessing a life-threatening event, such as combat, a natural disaster, a car accident, or sexual assault. PTSD can emerge from a one-time trauma as well as multiple traumatic events. However derived, PTSD left undiagnosed and untreated can lead to severe developmental or mental disorders costly to individuals, families and society (Ai, et al., 2013). What is more, early life trauma exposure may sensitize young children and place them at risk for internalizing or externalizing problems when exposed to subsequent, non-traumatic life stressors (Grasso et al, 2013).

Psychological assessment of adults and children for PTSD and other mental disorders is often done via face to face interviews or via paper checklists filled out by patients wherein patients are asked to describe their symptoms and histories. These questionnaires or checklists of symptoms are used to determine the presence or intensity of certain psychological or physiological symptoms. However, accurately assessing symptoms gleaned from anecdotal or self-reported symptoms as found in written or verbally administered questionnaires to adults and children suffering from PTSD can be a challenging, lengthy, inexact and expensive process.

During psychological assessment, non-trauma-based psychological disorders must be separated from trauma-based disorders in order that a correct and effective treatment plan can be devised for the patient. It can be difficult to plan treatment, however, when post-traumatic symptoms that are presented can be and often are misclassified as personality disorders or psychoses. Intrusive post traumatic symptoms may appear to be hallucinations or obsessions, and dissociative symptoms can lead to an incorrect diagnosis of schizophrenia. Post traumatic disorder symptoms of impulsivity, “acting out,” and lack of concentration can be mistakenly assessed as solely a result of, for example, borderline personality disorder or ADHD. Trauma-based cognitive symptoms can be incorrectly described as evidence for paranoia or other delusional processes (Briere, 1997).

Further, though findings indicate that nearly 72% of young children have experienced one or more types of traumatic events, very young children have limited capability to convey precise autobiographical details around the sources of their distress in ways that mental health workers can readily understand. As Scheeringa and Haslet (2010)[iv] note, “there is little reason to believe that children younger than 5 years would have sufficient skills to report their symptoms, and there have been no known studies with children younger than 7 years on their accuracy to self-report in relation to diagnoses.

Because self-reports of children under 7 years old are considered unreliable, assessments of disorders in young children with current technologies are therefore practically dependent on interviews of their caregivers. In fact, the vast majority of infant and toddler development screening programs in public and private preschools as well as Head Start use the Ages and Stages Questionnaire (ASQ) and Ages and Stages Questionnaires: Social-Emotional (ASQ-SE) or PEDS questionnaires filled out by parents and sometimes teachers or caregivers. (http://www.pedstest.com/ComparisonofPEDSToolsandASQTools.aspx). ASQ and ASQ-SE surveys are administered at regular intervals to the parent or guardian reporting on their child's growth and development. Similarly, the list of young child mental health screening tools posted on the American Society of Pediatrics web site consists in its entirety of questions for parents (https://www.aap.org/en-us/advocacy-and-policy/aap-health-initiatives/Mental-Health/Documents/MH_ScreeningChart.pdf).

Though other observational screening tests have been used, these have not been popular in pre-kindergarten as well as primary care due to test length, time constraints, and difficulty managing children's behavior while the test is given (Glascoe & Marks, 2011). Commonly used observational scales looking at development are limited in scope and expensive to conduct. For example, the Minneapolis Preschool Screening Instrument-Revised (MPSI-R) only screens for sociability in the social-emotional dimensions, and requires 15-25 minutes of one-on-one screening time to implement and evaluate. Most of these assessments also need to be conducted by a trained paraprofessional. In addition, most of these surveys are not designed to screen for social and emotional problems including anxiety, attachment disorders, or PTSD. Further, it is also concerning that, in any event and circumstance, using parent screening questionnaires has been found problematic in their ability to accurately assess psychological and developmental disorders in children.

The U.S. Preventive Services Task Force (USPSTF), an independent, volunteer panel of national experts in disease prevention and evidence-based medicine found inadequate evidence on the accuracy of surveillance (active monitoring) by primary care clinicians to identify children for further evaluation for speech and language delays and disorders (which can be induced among other causes by psychological trauma effects). Further, as the Substance Abuse and Mental Health Services Administration (SAMHSA) advises, these caregiver-completed assessments should not be the primary assessment tool/component. With this population, the agency explains, there is general consensus in the field that caregivers are frequently not adequate reporters with regard to complex trauma for a variety of reasons, including the caregivers' own trauma histories that may lead them to either normalize certain behaviors or be triggered by behaviors that might be typical given the child's age. Also, given that one of the most persistent and prevalent responses to trauma is avoidance of triggers and reminders, caregivers with trauma histories could be very likely to avoid thinking about the impact of trauma on their child as well as its signs and indicators. Thus, the agency recommends that caregiver report assessment tools should be used to supplement the information gathered, and should not be used in lieu of a behavioral observation and a full biopsychosocial interview.

Similarly, for adult sufferers of toxic trauma and PTSD, traditional interviews and/or questionnaires may not adequately reveal trauma symptoms. Adults may hesitate to disclose what they feel are shameful or illegal details of their past actions, or due to their condition, they may simply not be able to consciously recall details or entire incidents of their history. In addition, because of national origin or various issues germane to case-by-case ethnicities or demographics, there may be language barriers that prevent full comprehension of questions posed by a clinician. Adult male veterans with PTSD may refrain from seeking help for their psychological problems entirely due to their fear of the social stigma resulting from any kind of involvement or engagement with the traditional mental health field.

In further threats to validity of assessment, some biometric assessment systems for PTSD and other dysfunctional mental conditions require the attachment of EEG or galvanic sensors to the face, head, hands, etc. of the user. These mechanical attachments can obscure accurate assessment of PTSD and other conditions since these devices on the body may be experienced as invasive and contribute to a reminder of the past trauma incident or related traumatic incidents.

The present invention uses an invisible, tablet-based projector, interface and camera system to apply stimuli and to capture all relevant biometrics including Heart Rate Variability (HRV), so there is no test-induced invasive component that could threaten the collection of accurate data by adding to or otherwise affecting the tested patient's physiological reactivity quotient. Sensors of the present invention are invisible and entirely transparent to the user so there is also no sense on the part of the user that the user is being recorded, a factor which can also potentially impair the authenticity of the data that is collected. Further, the present invention's metrics are taken from a robust literature and research on defensive reactivity rather than being drawn from a so-called “biometric template of a person” (where repeat sessions would be necessary as opposed to the present invention's single required session), and where it would be difficult to reliably posit the so-called “objective mental state” of the person as a valid baseline. In addition, the use of standardized, research-based valenced stimuli may be a more objective standard than what may be arbitrary or situational “individual stressors” or “material from therapy sessions” as per the metrics applied by other extent patents and patent applications.

The present invention seeks to provide a solution to current and prior invasive, non-reliable, expensive face-to-face portable systems testing for disturbances in ordinary mental functions. The present invention locates and cross-validates two or more variables induced by valenced pictorial stimuli and associated with measured heart rate, facial expression, trauma play scale movements, eye and gaze movement and patterns and ocular variables (pupil motility, pupil vergence, blink reflex) across a timeline. The present invention then reports on the complete consolidated biometric information captured by this integrated sensor system. The present invention's apparatus, system, and method of collecting, integrating and analyzing biometric defensive reactivity and other pathological biometric data is superior to current unassisted human observation and analytical methods.

The present invention, referred to herein as “Synapstory,” removes sources of subjective assessment and assessor bias, and assessment-based social stigma by employing biometric data elicited by stimuli from valenced pictorial icons within tablet-based games. “Synapstory” is a novel and proprietary Artificial Intelligence (AI) algorithm used “on the fly” to non-invasively gather undifferentiated reactivity data and then locate outlier scores on the basis of age- and gender-based normative data. The algorithm then compiles the evidence-based constructs into identified symptomatology clusters that singly or together mark certain neurological and/or psychological conditions including: Post Traumatic Stress Disorder (PTSD) or traumatic exposure effect; Autism Spectrum Disorder (ASD); Attention Deficit Disorder (ADHD); Traumatic Brain Injury (TBI). The application then “red flags’ and reports out “at risk” scores for one or more of the neurological or psychological conditions. Synapstory's Artificial Intelligence (AI) nature lies in its ability, as more and more individual cases and their data are input into the system, to perform increasingly precise “red flagging” of conditions based on their secondary, accompanying constructs, and also to increase system discriminant validity with regard to apparently similar neurological and psychological conditions. An example of a formation of constructs and symptomatology clusters is shown in FIG. 3.

Outlier reactivity data (e.g., scores higher than 2 standard deviations (SD) from average) can be generated by emotional stimuli as well as by perceptual or physiological deficits (as in the case of ASD, ADHD, or TBI, for example). In order to differentiate psychological condition reactivity data from neurological condition reactivity data, it becomes important to identify the stimulus or stimuli that generates the reactivity data. What distinguishes PTSD/traumatic exposure effect data from other data is that it is generated from “emotional stimuli.”

Emotional stimuli literature has shown that emotional faces depicting anger and fear in particular function as “threat stimuli” for participants in a variety of age groups and both genders. The “threat stimuli” automatically activate biological survival circuits which consist in a cluster of autonomic reactions in the body which Lang and Bradley collectively term “defensive reactivity.” Participants who suffer from PTSD, for example, or those who have trauma exposure effect, react to these threat stimuli with a statistically significantly higher or lower than average amount and degree of defensive reactivity. Pupil size and movement, eyelid speed and amount of movements, gaze fixation, avoidance, and tracking, and large and small muscle movement in the body can all be measured at very subtle levels of scale. The sum of autonomic reactivity to threat stimuli is called the participant's degree of defensive reactivity.

A “defensive reactivity construct” is described below. Though single physiological measures such as duration, location and size can be calculated, it is possible to combine certain of these singular measures into more meaningful “defensive reactivity constructs” based on validated studies in the literature. For example, the fact that study subjects with PTSD or those who have trauma exposure effect have been found to either attend preferentially to threat stimuli or avoid threat stimuli has long been an apparent and puzzling contradiction both found and posed in literature in the field. However, more recent literature has noted that when gaze is broken down into component parts, and examined on a more granular level and over a longer duration, the apparent contradiction resolves. Closer and lengthier examination reveals that PTSD threat stimulus behavior is characterized neither by simple avoidance or fixation, but rather with a fluctuating gaze pattern, meaning that the participant with trauma exposure effect will characteristically gaze at the threat stimulus at some point, look away from the stimulus for a measure of time, look back at the stimulus, etc. in a pattern found to be characteristic of PTSD and that has been deemed Attention Bias Variability (ABV). ABV is an example of a Synapstory defensive reactivity “construct” and one of several evidence-based constructs within the “Symptomatology cluster” that mark the PTSD “condition.”

Synapstory identifies key constructs and arranges them into symptomatology clusters ranked first by a marker or markers and then by most critical accompanying constructs. For example, in a Synapstory session where the participant generated data embodied by the ABV construct and also second tier constructs including dilation of the Platysma muscle (a “fear” response captured and formulated by the evidence-based “Noldus” facial expression capturing application which is synchronized to the Tobii eye gaze and movement software), Synapstory reports out the indicated PTSD symptomatology cluster.

Neurological reactivity constructs are described below. Similar to “high anxiety,” ASD, ADHD and TBI are conditions which share certain constructs with the PTSD symptomatology cluster so that identifying and “red flagging” the accurate symptomatology cluster at study could conceivably be problematic. However, these conditions separately and together comprise separate and distinctive constructs as shown below in Table 1. While all four conditions may be characterized by unique saccadic movements, pupillary speed and movement and HRV, for example, the individual condition's level, timing, and accompanying secondary variables and constructs vary in critical ways. Synapstory's granular and multi-dimensional level of analysis (i.e., the comprehensive range and scope of non-invasive reactivity sensors and data sources) reveals the different construct patterns among and within the conditions as well as their markers. In sum, the Synapstory algorithm sorts out, identifies, ranks and compiles the evidence-based constructs from consistent, reliable, granular-level data, into the accurate evidence-based symptomatology constructs before reporting them out.

Table 1 displays examples of both shared and divergent psychological and neurological conditions and their divergent constructs.

TABLE 1

| | | Gaze | | Gaze | | | | | | |
|---|---|---|---|---|---|---|---|---|---|---|
| | | (attention) | | (attention) | Gaze | | | | Eye | |
| | | Sustained | | Attention | (saccades) | Gaze | | | (anatomical) | |
| | | attention | | Bias | Higher | (saccades) | | | Pupillary | |
| | | on | | Variability | rate of | Slower | | | speed | |
| | Gaze | pleasant | | (ABV) | micro- | saccadic | | | movement | |
| | (attention) | scenes in | Gaze | Sustained | saccades | reaction | Gaze | Eye | deficits | Eye |
| | Impaired | pleasant- | (attention) | attention | around | time to a | (pursuit) | (anatomical) | (post | (anatomical) |
| | sustained | neutral | Attention | on threat | stimulus | disappearing | Abnormality | Pupil | emotional | Pupillary |
| | attention | pairs | deficit | stimuli | onset | object | in pursuit | vergence | stimuli) | vergence |
| PTSD | X | | | X | | | | | X | X |
| ASD | X | | X | | | X | X | | | |
| ADHD | X | X | | | X | X | | X | | |
| TBI | | | X | | | | X | | | |

The present invention includes a tablet game that is informed by a proprietary algorithm that computes, via the game, critical biometric and/or behavioral measures of the neurological and psychological conditions PTSD/trauma exposure effect, ASD, ADHD, and TBI. A score signaling that the user has met or exceeded the standard eye movements and ocular behaviors and toxic trauma exposure effect threshold “red flags” the user for further examination by a licensed psychological professional. The licensed professional can then determine by standard, validated psychological instruments and interviews whether or not and to what extent the individual has PTSD/trauma exposure effect, ASD, ADHD, and TBI.

According to a motivational theory of emotion (e.g., Bradley & Lang, 2000; Lang & Bradley, 2010; Lang & Davis, 2006), affects are prompted by the activation of limbic survival circuits. These “circuits” “tune” sensory systems, increase attention and perceptual processing, and mobilize the individual for action in the face of a “threat.” The activation of these survival circuits consists in a cluster of autonomic reactions in the body which Lang and Bradley collectively term “defensive reactivity.” Defensive reactivity occurs as components of the autonomic nervous system default to a “survival mode which mobilize internal ‘fight or flight’ mechanisms” as part of a natural defense mechanism aimed at ensuring survival.

“Defensive reactivity” (Lang, Bradley, 2000) describes how, during extreme threat, the autonomic system mobilizes in a fight or flight survival response. The ongoing complex of psychological and physiological effects or symptoms concomitant with limbic survival circuits tune sensory systems, increase attention and perceptual processing, and mobilize the subject for action. These “fight or flight” symptoms per se are often not consciously experienced by the subject who remains consciously unaware of them. Therefore, communications or disclosures about these survival states most often cannot be initiated to others in the subject's environment or environments. Therefore, those interacting with the subject in daily life will not find the patient's triggered state as either immediately identifiable nor apparent as belonging to a post-traumatic stress syndrome.

Left untreated, the complex of responses becomes part of a chronic post-traumatic stress syndrome where the autonomic reactions are automatically triggered and mobilized for action in response to a most often innocuous event that the mind and body have interpreted as an imminent threat. Research shows that pictorial stimuli have a triggering effect in the case of previous traumatic exposure to persons and events (Wangelin et al, 2011). The present invention uses research-based valenced pictorial stimuli that automatically trigger autonomic reactions which accompanied the user's past traumatic exposure. These and other features of the invention will be more readily understood upon consideration of the attached drawings and of the following detailed description of those drawings and the presently-preferred and other embodiments of the invention.

Other objects, advantages and novel features of the present invention will become apparent from the following detailed description of one or more preferred embodiments when considered in conjunction with the accompanying drawings.

### BRIEF DESCRIPTION OF THE DRAWINGS

FIG. 1 is a block diagram depicting an integrated, real-time-biometric recording and information-analytics device and system configured in accordance with an embodiment of the invention.

FIG. 2 is a flow chart depicting a method for assessing autonomic nervous system reactivity levels in accordance with an embodiment of the invention.

FIG. 3 is a flow chart depicting an example of a formation of constructs and symptomatology clusters.

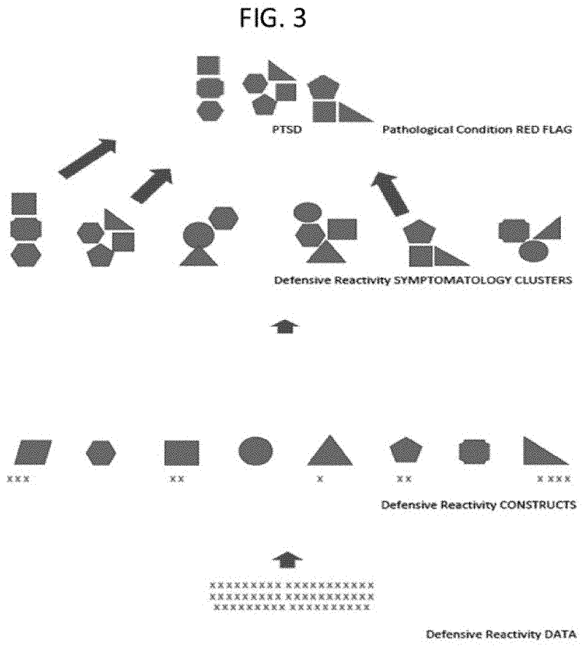

### DETAILED DESCRIPTION OF THE DRAWINGS

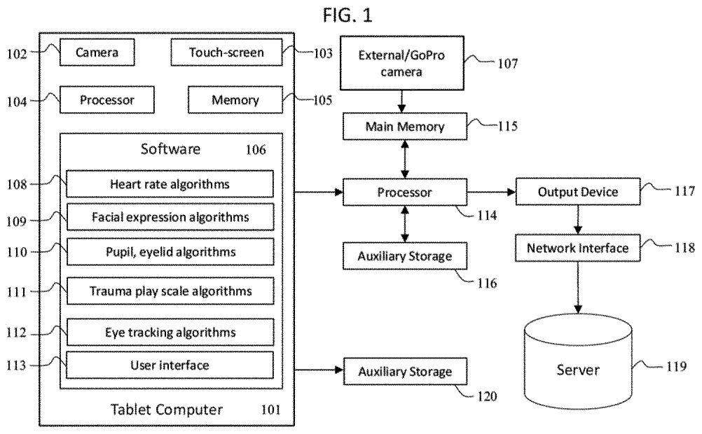

In FIG. 1, the integrated, real-time-biometric recording, and information-analytics device and system are illustrated as a block diagram of a representative digital tablet computer configuration which can perform the method of the invention.

In a presently preferred embodiment, the hardware platform includes a tablet computer 101 with a webcam 102, touchscreen 103, processor 104, memory 105, and software 106 enabling appropriate implementation and operations, algorithms, and calculations, as well as a connected external camera (e.g., GoPro camera) 107. FIG. 1 schematically illustrates exemplary software components housed in the tablet 101 including heart rate algorithms 108, facial expression algorithms 109, pupil and eyelid algorithms 110, trauma play scale algorithms 111, eye tracking algorithms 112, and a user interface 113 and integration. In one embodiment, the tablet computer 101 gathers the data via its camera 102 and processes all of the captured data on the processor 104 via the software 106 stored therein. The processed data can be displayed on the tablet or output externally, such as to another computer, an output device, a server or the like either directly or via a network.

In an alternative embodiment, as illustrated in FIG. 1, the system can include additional optional elements such as one or more of a processor 114, main memory storage 115, auxiliary storage 116, output device 117, network interface 118, and server 119. According to this embodiment, the tablet and the connected external camera can simply collect the data from the user (e.g., photos, videos and separate limbic survival circuit measures and/or scores) and output it to an external processing device, such as the external processor, output device, or server, either directly or via the network interface, without processing all of the data. Also, the tablet computer 101 can output its data directly to another auxiliary storage 120. Additionally, the system can insert session photos or videos of the user taken by the camera and then insert them among the pictorial stimuli shown to the user during the same session.

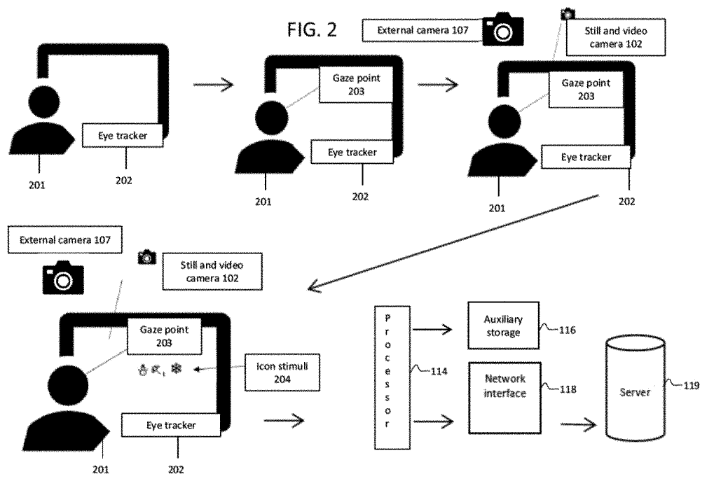

The processors described herein can be any type of processor, such as a field programmable gate array (FPGA), application specific integrated circuit (ASIC), central processing unit (CPU) and/or a microprocessor. The storage described herein can be any type of memory including random access memory (RAM), read-only memory (ROM), flash memory, a hard disk, a CD, a DVD, and/or cloud storage. The optional main memory and processor disposed outside of the tablet can be another computer, a server or the like. A specific example of a suitable hardware platform is a Microsoft PC Tablet running “Microsoft Windows 10,” but it is to be understood that the diagrams herein can be modified for other presently known operating systems (including Apple Incorporated's “IOS”), and other current or future hardware platforms. The software, described below, based on the flowchart shown in FIG. 2, is stored in a main memory and runs on a processor at run time, making use of the auxiliary storage and web server as needed.

Embodiments of the invention relate to an apparatus, method, and apparatus for evaluation of a subject's mental operations and/or physiological state from autonomic nervous system data and behavioral data. Specific embodiments involve evaluation of a subject's physiological state using devices that can determine the physiological state of a subject through the measurement and analysis of the subject's autonomous nervous system data and behavioral data including user's posture and distance from tablet. A specific embodiment relates to a device capable of capturing a subject's physiological and behavioral data, and then correlating and analyzing the data to provide an assessment of a subject's physiological state in regard to a specific dysfunction (e.g., TBI, autism) and/or defensive reactivity (e.g., PTSD) level in real time.

FIG. 2 is a flow-chart depicting a method for assessing autonomic nervous system reactivity levels in accordance with an embodiment of the invention. FIG. 2 illustrates a method for assessing mental dysfunction and/or defensive reactivity levels of a user using eye movement, heart rate, facial expression, ocular measures (pupil motility, pupil vergence, blink reflex) and play scale including body posture and distance from tablet data over time in conjunction with a timed series of valenced icon stimuli presented to the user, in accordance with one embodiment of the present invention.

As illustrated in FIG. 2, an eye tracker 202 includes a projector and a camera (e.g., web camera 102 of the tablet computer 101 of FIG. 1) in, for example, a tablet computer. The camera captures high-resolution photographs and videos of the face of the user 201 and takes high frame rate images of the user's eyes and gaze pattern. The projector creates a light of near infrared on the user's eyes. The external camera 107 captures synchronized video for the play scale, including user's posture and distance from the tablet 101.

The user's gaze point 203 is tracked by the eye tracker 202, and a web camera 102 continuously captures the facial expressions and heart rate of the user 201. Pictorial stimuli icons 204 are presented to the user on the touchscreen in order to elicit facial and eye responses and heart rate from the user which are captured by the camera. Data from sensors including, for example, the touch screen, web camera and eye tracker (projector, camera, and algorithms) and an external camera is collected and transmitted to the processor 114 where TBI, ADHD, ASD and defensive reactivity algorithms sum and score the eye, heart and facial expression and trauma data into separate scores relative to TBI, ADHD, ASD and/or defensive reactivity.

Autonomic reactivity data is gathered from the sensors, including at least gaze data, eye anatomy data, facial muscle data, and heart rate data. Within each data category, a distinction can be made between a normal neurological response and defensive reactivity. From the normal neurological response, outlier neurological constructs, including marker constructs, and accompanying constructs can be used to determine/flag, for example, a neurological symptomatology cluster X with a marker and one or more secondary constructs to determine a neurology condition. Likewise, the defensive reactivity data can be used with outlier defensive reactivity construct clusters, marker constructs, and accompanying constructs to determine/flag a variety of neurological conditions. For example, a neurological symptomatology cluster X with a marker and a secondary construct can be used to determine a neurology condition, or a defensive symptomatology cluster X with a marker and one or more secondary constructs to determine a defensive reactivity condition.

Additionally, the eye anatomy data, the facial muscle data, and the heart rate data can be used alone or in any appropriate combination to determine/flag neurological symptomatology clusters and/or defensive symptomatology clusters as described above in relation to the gaze data. For example, pediatric TBI with ADHD can be identified based on neurological symptomatology marker(s) and accompanying construct(s). As another example, Complex PTSD can be identified based on defensive reactivity condition(s) with particular markers and secondary constructs.

The data from the processor 114 is stored in auxiliary storage 116 and transmitted to the network interface 118 which links the computer 101 to the private network. Through the network interface 118, the data can be sent to a database and the server 119.

According to an exemplary embodiment of the invention, there is provided a non-transitory computer-readable medium encoded with a computer program for performing the above-described operations. The term “computer-readable medium” as used herein refers to any medium that participates in providing instructions for execution. Common forms of computer-readable media include, for example, a floppy disk, a flexible disk, a hard disk, magnetic tape, any other magnetic medium, a CD-ROM, any other optical medium, punch cards, paper tape, any other physical medium with patterns of holes, a RAM, a PROM, an EPROM, a FLASH-EPROM, any other memory chip or cartridge, and any other non-transitory medium from which a computer can read.

The following are merely examples of some of the objects achieved by the embodiments of the invention:

To track types and degrees of intensity of stimuli to nervous system and behavioral reactions through biometric sensor data and valenced icon interaction analytics;

To improve inferential discrimination among different types and severity of mental dysfunction including PTSD, ADHD, TBI and ASD through objective measurements and longitudinal information of higher specificity and accuracy;

To improve diagnosis and patient monitoring;

To capture objective measurements of dynamic and/or static on-screen content, or stress-inducing on-screen stimuli through event monitoring device systems and develop a comprehensive patient illness condition;

To provide patient feedback and visualization of objective comparisons of dysfunction or illness progress between successive visits;

To create and provide real time evidence-based measurable and objective inter- and intra-patient longitudinal information to the physicians or mental health workers and therapists in mental healthcare treating the patient for the first time. This facilitates the primary care physicians' and specialists' development, employment and deployment of targeted protocol and assessment to and for the patient.

To provide for a quantitative comparison of changes between the initial, subsequent, and successive sets of biometric data in terms of one or more of frequency, duration, intensity, deviations, and summary statistics of a single user regarding targeted icons in order to improve specificity for clinician diagnosis over pre- and post-treatment therapy sessions; and potentially to categorize into low, medium, or high severity levels for each mental dysfunction type suggested by the invention.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method of determining a mental health state, comprising:
outputting pictorial stimuli to a user of a user interface;
measuring, in response to the pictorial stimuli, a plurality of physical values of the user, including at least one of a heart rate, a facial expression, a trauma play scale movement, an eye movement, and an ocular variable including at least one of a pupil motility, a pupil vergence, and a blink reflex;
cross-validating the plurality of physical values across a timeline;
determining the mental health state of the user based on the cross-validated plurality of physical values; and
outputting a diagnosis of the user based on the mental health state of the user.

2. The method according to claim 1, wherein the pictorial stimuli are presented to the user subliminally while the user is engaged in another activity with the user interface.

3. The method according to claim 2, wherein the another activity includes playing an interactive computer game.

4. The method according to claim 1, wherein the plurality of physical values are measured by an image sensor.

5. The method according to claim 1, wherein the method includes determining whether the user has post-traumatic stress disorder by cross-validating across a valenced icon stimulus timeline plus two or more values associated with measured eye movements and ocular variations including at least one of pupil motility, pupil vergence, and blink reflex.

6. The method according to claim 1, wherein the method includes determining whether the user has a brain injury by detecting at least one of a pattern of eye movements and a heart rate of the user.

7. The method according to claim 1, wherein the method includes determining whether the user has autism spectrum disorder by detecting changes or marked discontinuities in at least one of eye movement over time, ocular behavior over time, and heart rate of the user.

8. The method according to claim 6, wherein detecting changes or marked discontinuities in at least one of eye movement and ocular behavior over time of the user includes at least one of impaired sustained attention on the pictorial stimuli, attention deficit, a saccadic reaction time to a disappearing object in the pictorial stimuli, and an abnormality in following a moving object in the pictorial stimuli.

9. The method according to claim 1, wherein the method includes determining whether the user has attention-deficit hyperactivity disorder by detecting changes or marked discontinuities in at least one of eye movement and ocular behavior over time of the user.

10. The method according to claim 8, wherein detecting changes or marked discontinuities in at least one of eye movement and ocular behavior over time of the user includes at least one of impaired sustained attention on the pictorial stimuli, sustained attention on pleasant scenes in pleasant-neutral pairs in the pictorial stimuli, a rate of micro-saccades around stimulus onset, a saccadic reaction time to a disappearing object in the pictorial stimuli, and pupil vergence.

11. A non-transitory computer-readable medium storing a program for determining a mental health state of user of a user interface which, when executed, causes a processor to:
output pictorial stimuli to the user of the user interface;
measure, in response to the pictorial stimuli, a plurality of physical values of the user, including at least one of a heart rate, a facial expression, a trauma play scale movement, an eye movement, and an ocular variable including at least one of a pupil motility, a pupil size or vergence, and a blink reflex;
cross-validate the plurality of physical values across a timeline;
determine the mental health state of the user based on the cross-validated plurality of physical values; and
output a diagnosis of the user based on the mental health state of the user.

12. The non-transitory computer-readable medium according to claim 11, wherein the pictorial stimuli are presented to the user subliminally while the user is engaged in another activity with the user interface.

13. The non-transitory computer-readable medium according to claim 12, wherein the another activity includes playing an interactive computer game.

14. The non-transitory computer-readable medium according to claim 11, wherein the plurality of physical values are measured by an image sensor.

15. The non-transitory computer-readable medium according to claim 11, wherein the program causes the processor to determine whether the user has post-traumatic stress disorder by cross-validating across a valenced icon stimulus timeline plus two or more values associated with measured eye movements and ocular variations including at least one of a pupil motility, a pupil size or vergence, and a blink reflex.

16. The non-transitory computer-readable medium according to claim 11, wherein the program causes the processor to determine whether the user has a brain injury by detecting a pattern of eye movements of the user.

17. The non-transitory computer-readable medium according to claim 11, wherein the program causes the processor to determine whether the user has autism spectrum disorder by detecting changes or marked discontinuities in at least one of eye movement and ocular behavior over time of the user.

18. The non-transitory computer-readable medium according to claim 17, wherein detecting changes or marked discontinuities in at least one of eye movement and ocular behavior over time of the user includes at least one of impaired sustained attention on the pictorial stimuli, attention deficit, a saccadic reaction time to a disappearing object in the pictorial stimuli, and an abnormality in following a moving object in the pictorial stimuli.

19. The non-transitory computer-readable medium according to claim 11, wherein the program causes the processor to determine whether the user has attention-deficit hyperactivity disorder by detecting changes or marked discontinuities in at least one of eye movement and ocular behavior over time of the user.

20. An apparatus for determining a mental health state of a user, comprising:
a user interface configured to display pictorial stimuli to the user;
an image sensor configured to measure, in response to the pictorial stimuli, a plurality of physical values of the user, including at least one of a heart rate, a facial expression, a trauma play scale movement, an eye movement, and an ocular variable including at least one of a pupil motility, a pupil size or vergence, and a blink reflex; and
a processor configured to cross-validate the plurality of physical values across a timeline and determine the mental health state of the user based on the cross-validated plurality of physical values;
wherein the user interface is configured to output a diagnosis of the user based on the mental health state of the user.
